(19) United States
(12) Patent Application Publication
(10) Pub. No.: US 2004/0267204 A1
(43) Pub. Date: Dec. 30, 2004

(54) On-demand needle retaining and locking mechanism for use in intravenous catheter assemblies

(76) Inventor: Robert M. Brustowicz, Waban, MA (US)

Correspondence Address: David Prashker, DAVID PRASHKER, P.C., P.O. Box 5387, Magnolia, MA, 01930, US

(21) Appl. No.: 10/606,554

(22) Filed: Jun. 26, 2003

(51) Int. Cl. A61M 5/178

(52) U.S. Cl. 604/168.01

(57) ABSTRACT

The present invention provides a structure and mechanism for retaining and locking a piercing needle in a non-movable position on-demand while positioned co-axially within a hollow cannula in an intravenous catheter assembly. The needle restraining mechanism prevents accidental and inadvertent rearward movement of the piercing needle during the initial and any subsequent attempts to pierce and intravenously cannulate a blood vessel successfully in-vivo.

### FIELD OF THE INVENTION

The present invention is concerned generally with intravenous catheterization methods and intravenous catheter assemblies; and is particularly directed to structures and mechanisms to prevent premature withdrawal of the puncturing needle into the safety chamber of an intravenous catheter apparatus if a blood vessel was not cannulated successfully on the first attempt at insertion of a needle-catheter assembly.

### BACKGROUND OF THE INVENTION

Intravenous catheters for the infusion of fluids into the peripheral veins of a patient are one of the most common devices used in intravenous therapy. Such intravenous catheter assemblies are typically produced in two general forms: through-the-needle catheters, in which a catheter is threaded through the needle cannula and into the vein of a patient; and over-the-needle catheters, in which the needle and concentric outer catheter are inserted into the vein and the needle is withdrawn through the emplaced catheter.

A typical over-the-needle intravenous catheter assembly requires the user to remove and then dispose of a contaminated needle after the catheter is properly located in a blood vessel of a patient. A typical catheter design effective for this purpose is described by U.S. Pat. No. 4,762,516. This catheter apparatus includes an elongate body which houses a sliding needle guard. In use, the needle with its surrounding catheter tube is inserted through the skin of a patient until the tip of the needle is located in a blood vessel, a position detected by a small flow of blood through the needle and into the flash chamber of the catheter. The user then advances a tab on the top of the needle guard to simultaneously thread the catheter tube into the blood vessel and begin the retraction of the needle from within the catheter tube. As the needle is withdrawn rearward from the emplaced catheter, the forward advance of the tab slides the needle guard backward out of the housing until the distal end of the needle tip becomes enclosed and the proximal end of the needle guard becomes locked within the elongate body. The needle and needle guard may then be set aside with the needle tip fully protected. Once the needle is withdrawn from the catheter, the user's immediate priorities are infusion set connection and site preparation, including the taping of the catheter to the patient.

Merely representative of these protective articles for catheter insertion devices are: the catheter with needle guard described by U.S. Pat. No. 5,000,740; the catheter with needle gasket revealed by U.S. Pat. No. 5,092,845; the hypodermic needle safety shield of U.S. Pat. No. 5,152,751; the safety disposable intravenous I.V. assembly illustrated by U.S. Pat. No. 5,205,829; the intravenous catheter with automatically retracting needle-guide of U.S. Pat. No. 5,562,634; the racketing needle protector assembly exemplified by U.S. Pat. No. 5,725,503; the needle protector of U.S. Pat. No. 5,851,196; the catheter apparatus having valved catheter hub and needle protector described by U.S. Pat. No. 5,954,698; the self-contained safety intravenous catheter insertion device of U.S. Pat. No. 6,056,726; the catheter needle safety device illustrated by U.S. Pat. No. 6,117,110; the retractable hypodermic needle assembly shown by U.S. Pat. No. 6,302,868; the catheter insertion device with retractable needle of U.S. Pat. No. 6,436,070; and the tubular intravenous set described by U.S. Pat. No. 6,517,522.

There remains, however, a very different and long-standing problem for persons using conventional intravenous needle-catheter assemblies. When starting an intravenous flow line, the patient's blood vessel (typically a superficial vein of choice) is often not pierced by the needle on the initial attempt; and the user typically then pulls back on the catheter assembly in order to pull it out and reposition it for a second attempt. However, as the user pulls back on the elongated body of the catheter assembly in order to reposition it for a second attempt at piercing the vein, the pressure and friction of the patient's skin at the initial puncture site presses against the inserted catheter tip; and this pressure causes a marked clinging and holding of the inserted cannula tip in place. Thus, as the user attempts to withdraw the catheter, the needle portion of the apparatus typically becomes inadvertently drawn rearward and is pulled into the protective safety chamber of the assembly; and, if one is not extremely careful, the needle portion then becomes unavoidably locked-in-place within the protective safety chamber and subsequently cannot be moved forward again without great difficulty. As a consequence, because the now locked-in-place needle cannot be moved from within the protective safety chamber without a major effort and much inconvenience, the user routinely abandons his attempts to free the locked-in-place needle; pulls the original catheter assembly completely from the puncture site in the skin; and obtains and employs an entirely new and different, second intravenous catheter assembly for his second attempt to pierce and cannulate the chosen blood vessel in the patient.

For these reasons, it is commonly recognized in this technical field that conventional intravenous needle-catheter assemblies (although having a range of different protective safety chambers, enveloping shells and needle guard features for the piercing needle) are nevertheless seriously flawed in design, structurally inadequate, and functionally deficient. Clearly, there remains a substantive need and requirement for structures offering on-demand needle retaining mechanisms which provide the advantages of using a traditional intravenous catheter assembly with the functional operability and safety advantage of being able to withdraw the needle into a protective shell or chamber.

### SUMMARY OF THE INVENTION

The present invention has at least two different aspects and alternative structural formats.

A first aspect is an on-demand needle retaining and locking mechanism for use in an intravenous needle-catheter assembly, said mechanism comprising:

a rotable on-demand needle-safety container comprised of

(i) an elongated shell having at least one discrete wall and being of predetermined dimensions and configuration,

(ii) an open end in said shell adapted for passage there through of a piercing needle,

(iii) an internal spatial volume within said shell sufficient for containing and securing the entirety of a piercing needle,

(iv) a sized tab member disposed on an exterior surface of said shell at a prechosen aligned position adjacent to, but axially removed from, said open end, and

(v) a plurality of pre-positioned radial and axial cutouts in said wall of said shell, at least one of said cutouts being radially aligned with, but removed from, said tab member; and

a needle housing unit adapted for mounting upon and axial movement at will over said rotable needle-safety container, said needle housing unit being comprised of

(a) a casing of predetermined dimensions, configuration, and overall spatial volume,

(b) an optional configured spool section comprising a tab-engagement segment, and at least one sized notch for on-demand engagement with said tab member of said needle-safety container, said spool portion being alignable at will with and being able to engage, retain, and disengage said tab member of said rotable needle-safety container on-demand,

(c) an extended body section,

(d) a flash chamber for holding one end of a piercing needle, and

(e) a guide member for aligned movement radially and axially at will through said pre-positioned cutouts in said wall of said needle-safety container.

A second aspect is an on-demand needle retaining and locking mechanism for use in an intravenous needle-catheter assembly, said mechanism comprising:

a needle-safety container comprised of

a linear shell

(i) having at least one discrete wall and being of predetermined dimensions and configuration,

(ii) having an open end in said shell adapted for passage there through of a piercing needle, and

(iii) having an internal spatial volume sufficient for containing and securing a piercing needle,

(iv) at least one pre-positioned axial cutout in said wall of said linear shell,

a hollow collar contiguously aligned with and rotably attached to an open end of said linear shell, said rotable collar

(1) having at least one wall and being of predetermined dimensions and configuration,

(2) having two open ends adapted for passage there through of a piercing needle,

(3) a solid tab member which is disposed on an exterior surface of said wall, and

(4) at least one pre-positioned radial cutout in said wall which is radially positioned and aligned with, but spatially removed from, said solid tab member; and

a needle housing unit adapted for mounting upon and axial movement at will over said needle-safety container and said rotable collar, said needle housing unit being comprised of

(a) a casing of predetermined dimensions, configuration, and overall spatial volume,

(b) an optional configured spool section comprising a flanged rib and a tab-engagement segment, and at least one sized notch for on-demand engagement with said tab member of said rotable collar, said spool section being alignable at will with and being able to engage, retain and disengage said tab member of said rotable collar on-demand,

(c) an extended body section, and

(d) a flash chamber for holding one end of a piercing needle, and

(e) a guide member for aligned radial and axial movement at will through said pre-positioned cutout of said rotable collar and for aligned axial movement at will through said pre-positioned cutout of said linear shell of said needle-safety container.

### BRIEF DESCRIPTION OF THE FIGURES

The present invention may be better appreciated and more easily understood when taken in conjunction with the accompanying drawing, in which:

FIG. 1 is an overhead perspective view of an improved needle-catheter assembly incorporating the present invention;

FIG. 2 is a cross-sectional view of the improved needle-catheter assembly of FIG. 1;

FIG. 3 is an overhead perspective view of the needle-catheter assembly of FIGS. 1 and 2 prior to the placement of the hollow cannula and the catheter hub on the tapered needle guard tip;

FIG. 4 is an overhead perspective of the assembly of FIG. 3 after the mounted needle housing unit has been moved rearward over the axial length of the needle-safety container;

FIGS. 5A-5C are frontal, cross-sectional and bottom views of a first preferred embodiment for the needle-safety container;

FIGS. 6A-6C are frontal, cross-sectional and bottom views of a second preferred embodiment for the needle-safety container;

FIGS. 7A-7D are frontal, overhead, side, and bottom views of a preferred embodiment for the needle housing unit;

FIG. 8 is an overhead perspective view of the improved needle-catheter assembly incorporating the present invention when properly arranged for use;

FIG. 9 is an overhead perspective view of the improved needle-catheter assembly of FIG. 8 in a right-handed needle retaining and locking mode of use;

FIG. 10 is an overhead perspective view of the improved needle-catheter assembly of FIG. 8 in a left-handed needle retaining and locking mode of use; and

FIG. 11 is an overhead perspective view of the improved needle-catheter assembly after the needle retaining and locking mechanism has been disengaged and the needle retracted to its secured-in-place position.

### DETAILED DESCRIPTION OF THE INVENTION

The present invention is an on-demand needle retaining structure and locking mechanism which is intended to serve as a substantive part of and beneficial improvement for intravenous needle-catheter assemblies routinely employed for inserting an intravenous flow line for administration of fluids and medicaments. The present invention offers and provides major advantages and marked benefits for the user. These include the following:

1. The needle retaining structure allows the needle-catheter assembly to be radially rotated, either to the left or right; and then be locked on-demand so that the needle cannot slide accidentally into the protective safety chamber of the apparatus. The act of retaining the needle and locking the mechanism can therefore be accomplished at will by either a right-handed or a left-handed user without inconvenience, awkwardness, or loss of control.

2. The needle retaining and locking mechanism operates as a single unit. There are no intricate parts, no difficult manipulations, and no cumbersome features or structures to control. The invention is structurally simple and easy to operate.

3. The catheter assembly incorporating the needle retaining and locking structure can be grasped by the user naturally and held in position with the thumb and forefinger in a fashion which is very comfortable and similar to holding catheters not having such features. The present invention adds no weight or bulk to the catheter device; does not alter or modify essential needle-catheter functions; and requires no unusual skill or dexterity to operate successfully.

4. The needle retaining and locking structure can be engaged on-demand, i.e., at will. It can and should be engaged prior to the initial attempt to pierce and cannulate a blood vessel; be retained in locked position for a second or any multiple attempts to cannulate a vein; and then be disengaged after cannulation at will. Thus, if one is unsuccessful in cannulating the blood vessel initially, one can retract the entire assembly; and then, with the person's fingers in the same position, make a second effort or a greater number of attempts as many times as is necessary.

The present invention is best understood and most easily described in the context of an intravenous needle-catheter assembly. Accordingly, a complete and detailed disclosure of one preferred embodiment of such an assembly is provided below. It will be clearly understood and appreciated, however, that the needle-catheter apparatus as an article is conventionally known; may vary both in form and size; and is presented herein merely as a representative example and description illustration of those articles commonly employed today in this technical field.

### I. An Improved Intravenous Needle-Catheter Assembly

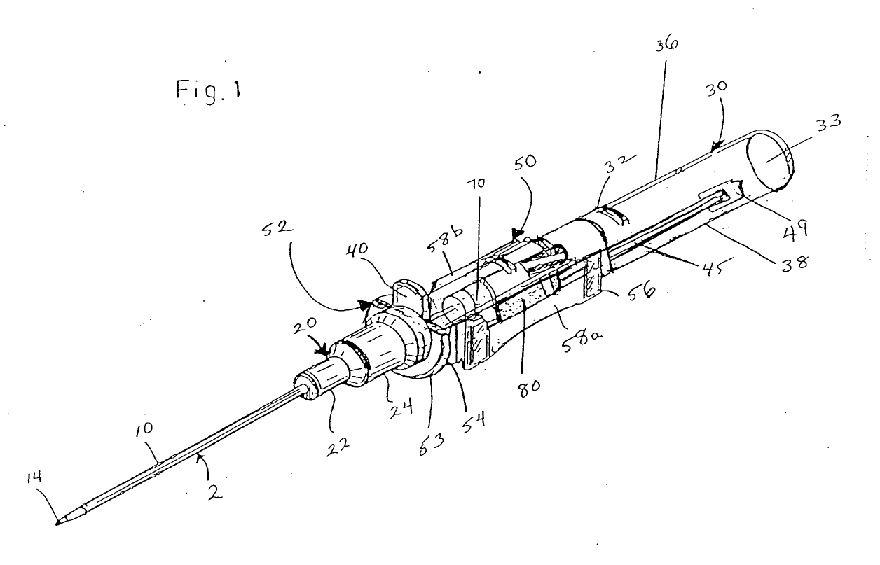

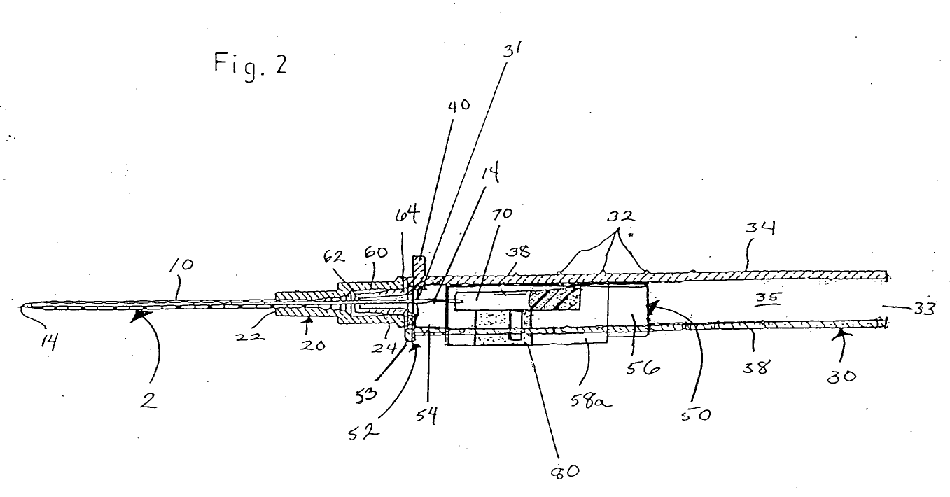

FIG. 1 shows an overhead, perspective view of an improved needle-catheter assembly which is constructed using and incorporates the present invention, while FIG. 2 shows a cross-sectional view of the same improved assembly. As illustrated collectively by FIGS. 1 and 2, the needle-catheter assembly 2 comprises: an elongated hollow cannula or catheter tube 10; a piercing needle 14 positioned co-axially within the internal lumen of an outer, concentric hollow cannula 10; a catheter hub 20 encompassing one end of the hollow cannula 10 and the piercing needle 14 concurrently; a cylindrically shaped needle-safety container 30 disposed adjacent to and aligned with the catheter hub 20; and a semi-tubular needle housing unit 50 which is open at the top, engages and supports one end (the proximal end) of the piercing needle 14, and is mounted upon and is slidable over the length of the cylindrically shaped needle-safety container 30.

The hollow cannula or catheter tube 10 extends from and is connected to the distal hub end 22 of the catheter hub 20 and is concentric therewith. The hollow cannula 10 is attached to the distal hub end 22 by any means presently known in the art, including adhesively or mechanically by means of a metal eyelet. The larger-diameter proximal hub portion 24 of the catheter hub 20 is flanged at one end for connection to any conventionally known infusion set suitable for delivery of fluids to the bloodstream; and the inner diameter of the proximal hub portion 24 of the catheter hub 20 is sized to fit over the distal end 62 of a tapered protective tip 60, which lies concentrically within the catheter hub 20. In similar fashion, the proximal end 64 of the tapered protective tip 60 is axially aligned with and is typically joined to one open end of the cylindrical needle-safety container 30 positioned directly adjacent thereto. Via this arrangement, the needle-safety container 30 lies disposed adjacent to and is axially aligned with the center of the catheter hub 20 as well, as with the co-axially positioned piercing needle 14 then lying within the lumen of the hollow cannula 10 such that an unobstructed, centrally located, and commonly shared communication flow pathway is generated by and through them collectively.

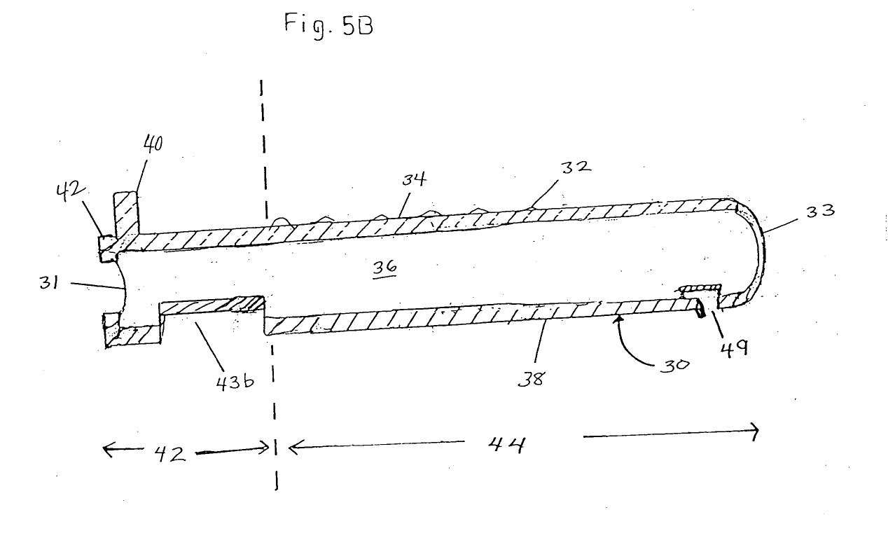

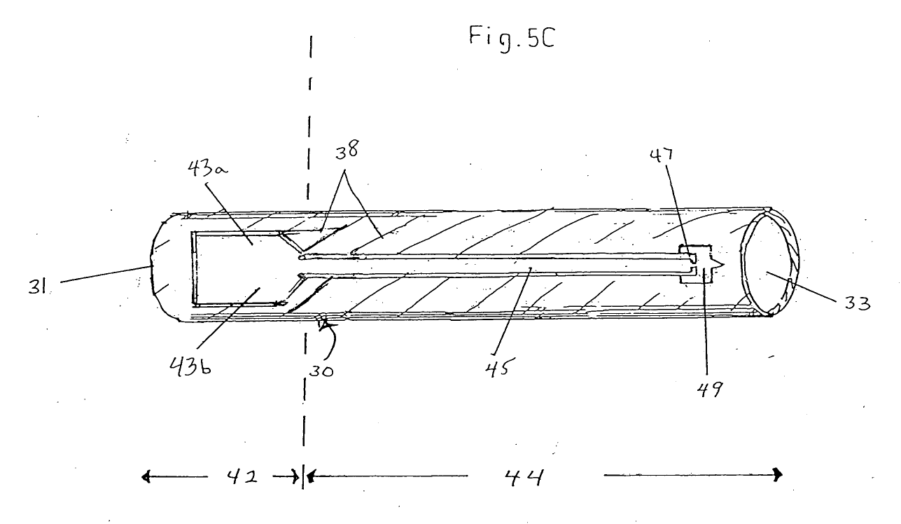

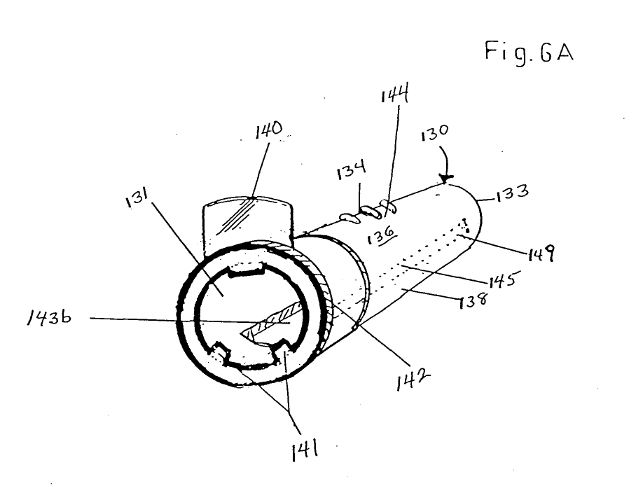

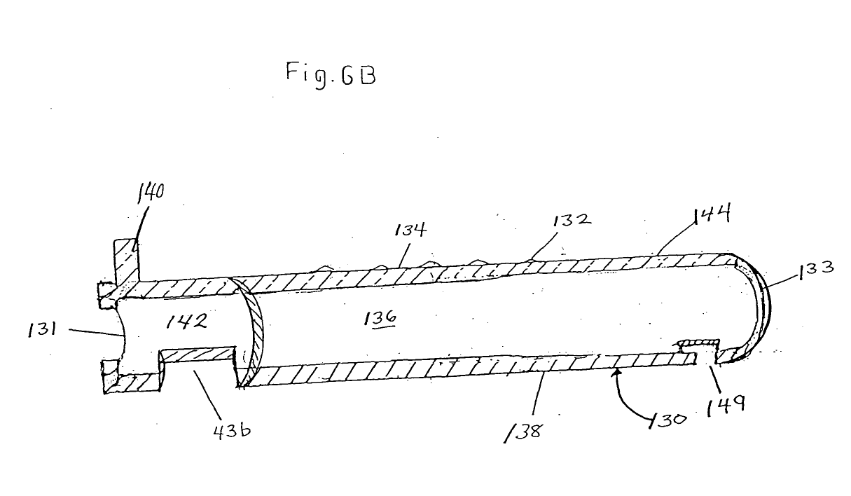

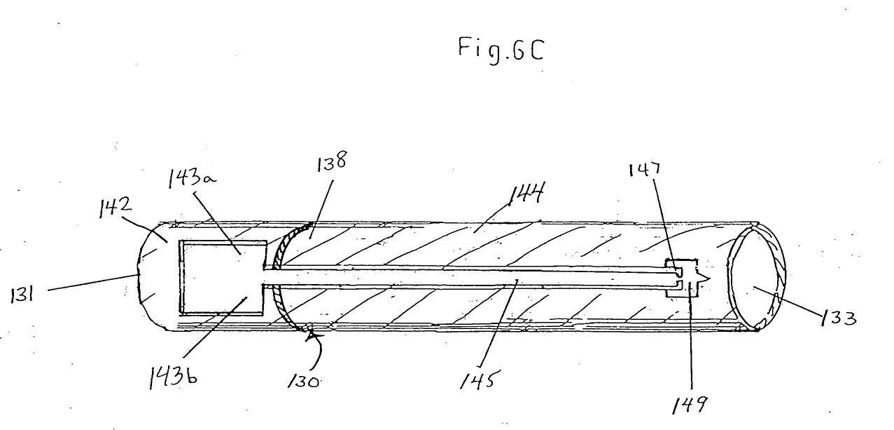

Also as seen in FIGS. 1 and 2 respectively, the needle-safety container 30 appears as an elongated shell (or hollow cylinder) which is placed axially along its length within the catheter assembly 2; and when so placed, presents at least one wall having discrete top, bottom and sidewall orientated surfaces. Near the distal end 31 of the shell comprising the needle-safety container 30 is a resilient solid tab member 40 which is employed as an essential component in the retaining structure and locking mechanism comprising the present invention. On the top surface 34 of the shell are a plurality of contoured projections 32 which are useful as finger friction grips when manipulating the catheter assembly 2. In contrast, the sidewall surfaces 36 and the bottom surface 38 of the shell are typically smooth. However, the bottom surface 38 of the shell is split and purposely cut to provide a series of pre-positioned, aligned and geometrically sized cutouts—which include a parallel pair of contoured slots, a narrow median groove, and an intersecting widened terminal end recess. These configured cutouts in the bottom surface as well as other particular structural features of the needle-safety container 30 are shown explicitly and in greater detail by FIGS. 5A-5C respectively, and appear in an alternative format by FIGS. 6A-6C respectively.

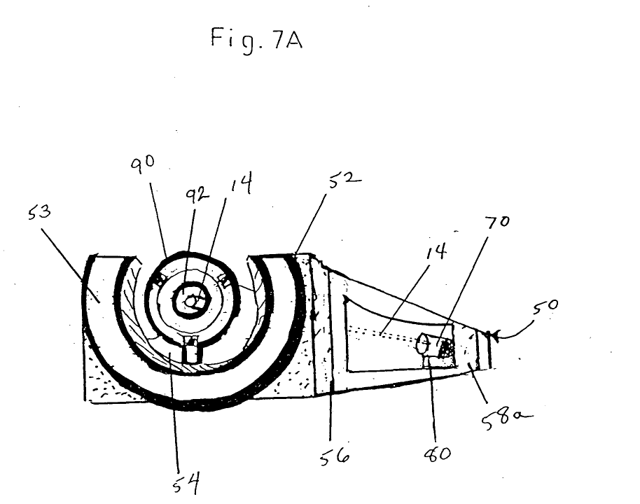

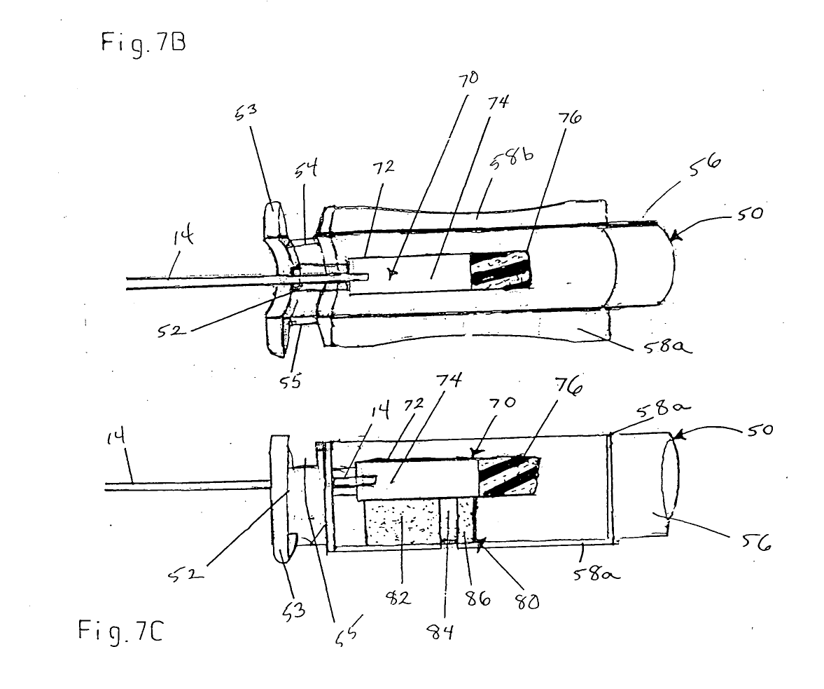

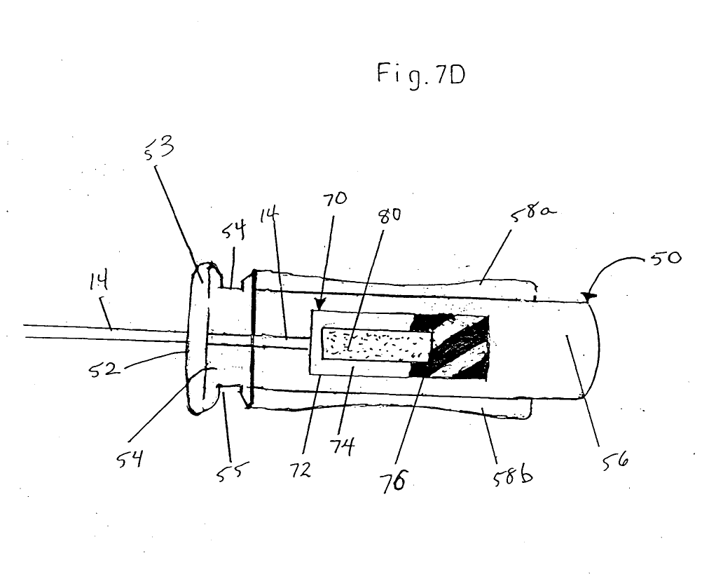

In addition, the needle-catheter assembly 2 includes a structurally modified needle housing unit 50 which provides an essential component of the present invention. The needle housing unit 50 is a configured casing mounted upon and extending within the needle-safety container 30; is slidable over the sidewall and bottom surfaces of the cylindrical shell; and typically is semi-tubular or U-shaped in overall configuration and thus commonly open at its top. These particulars and features are shown in detail by FIGS. 7A-7D respectively.

Thus, as shown by FIGS. 1, 2 and 7 collectively, the needle housing unit 50 is structurally constructed as a configured casing having predetermined dimensions and overall spatial volume; and comprises a spool section 52 having a semi-circular flange 53, a tab-engaging segment 54 (typically of reduced diameter with respect to the size of the flanged rib 53), and a pair of sized notches 55; as well as a discrete U-shaped extended body section 56 of sufficient size and volume to accommodate the fingers of the human hand. Attached to opposing sides of the extended body section 56 are a pair of molded side panels 58a and 58b which provide easy-grip surfaces for holding and sliding the entire needle housing unit 50 over the axial length of the needle-safety container 30 in either axial direction at will.

The semi-tubular U-shaped configuration of the extended body section 56 provides an interior spatial volume adequate to house several other features and elements. Positioned and contained within the interior volume of the needle housing unit 50 are a discrete flash chamber 70, a downwardly extending guide member 80, and a needle hub 90 (not shown in FIG. 2).

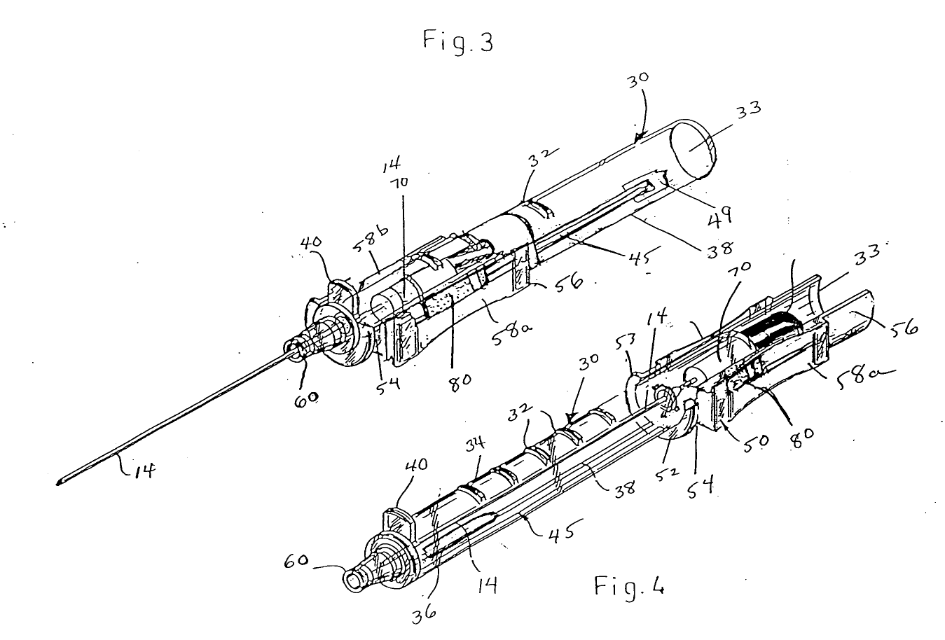

FIG. 3 shows the catheter assembly of FIGS. 1 and 2 in proper position prior to the placement of the hollow cannula and the catheter hub on the tapered needle guard tip 60. In this instance, almost the full axial length of the piercing needle 14 is externally exposed and directly visible to the naked eye; and its proximal end is shown as passing through the tapered protective tip 60 and being joined to the flash chamber 70 lying within the slidable needle housing unit 50 mounted upon and within the needle-safety container 30. Note also that, in this instance, the slidable needle housing unit 50 lies at its extreme forward location, is axially located immediately adjacent the tapered protective tip 60, and is available for movement rearward at will.

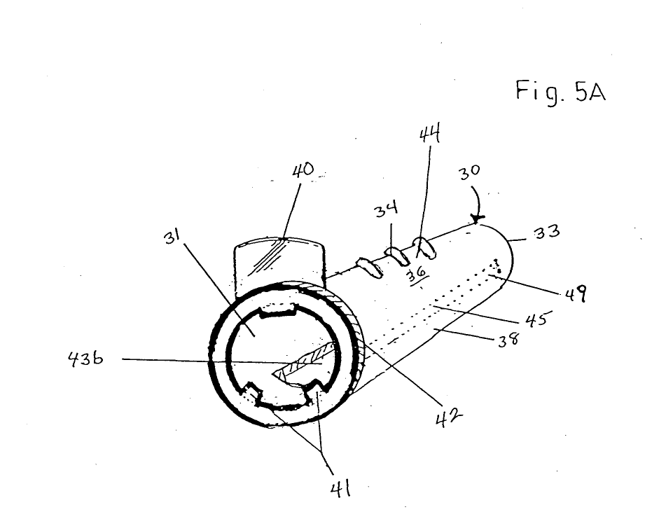

In contrast, FIG. 4 shows the catheter assembly 2 after the mounted needle housing unit 50 has been slided rearward over and along the axial length of the cylindrical needle-safety container 30 to its most rearward location, and now rests in a secured-in-place position. As shown therein, the needle housing unit 50 is axially located immediately adjacent the terminal end 33 of the cylindrical needle-safety container 30; and the piercing needle 14 has receded from its prior (externally exposed) location and has become internalized completely (and consequently thus lies entirely) within the interior spatial volume of the cylindrical needle-safety container 30. In this position, the downwardly extending guide member 80 (attached to both the flash chamber 70 and the body section 56) has become secured-in-place and lies fixed within the wide terminal end recess 49 existing in the bottom surface 38 of the needle-safety container 30. In this position, the needle housing 50 cannot be made movable again without a major effort by the use to free the guide member 80 physically from the secured-in-place location. This terminal fixed needle position for the catheter assembly 2 illustrates the conventionally known safety feature which prevents the piercing needle from becoming inadvertently exposed and being a danger to the user.

### II. The Structure Constituting the Needle Retaining and Locking Mechanism

The needle restraining mechanism comprising the present invention is provided by the presence and interaction of particular structural features present upon two different components of the apparatus: the sized tab member disposed upon the rotable needle-safety container; and the spool section of the slidable needle housing unit. Each of these will be described in detail below.

### A. The Needle-Safety Container Comprising a Rotable Tab Member:

### A FIRST PREFERRED EMBODIMENT OF THE NEEDLE-SAFETY CONTAINER

A first preferred embodiment of the needle-safety container is seen in FIGS. 5A, 5B and 5C respectively. FIG. 5A illustrates this article in a frontal, perspective view; FIG. 5B shows the shell container in a cross-sectional view; and FIG. 5C provides a bottom view of the structure. Clearly, FIGS. 5A-5C are to be considered collectively and cumulatively as a whole.

Accordingly, an elongated needle-safety container 30 appears as a hollow wall shell having at least one wall, predetermined dimensions, and a set configuration; and is intended to be used in a needle-catheter assembly when positioned axially along its length as shown by FIG. 5A. When placed in its intended axial position, the elongated wall of the needle-safety container 30 presents a top surface 34, two sidewall surfaces 36, and a bottom surface 38 as discrete entities; and provides an identifiable forward (distal) end 31, an internal lumen 35, and a terminal (proximal) end 33.

The forward end 31 shown in FIG. 5A is intended to be aligned to, engaged with and preferably joined to the tapered protective tip 60 of the needle-catheter assembly [see FIGS. 1 and 2]. In this embodiment, the forward end 31 presents and includes a trio of supporting ledges 41 which are disposed within the internal lumen of the shell container 30 and which are to hold an optional needle-supporting medial disc (not shown)—which is an optional feature typically employed to hold and support the length of the piercing needle 14 as it moves axially within the assembly 2. These supporting ledges 41 are an optional, but highly desirable feature which, in combination with the optional medial disk, will facilitate the operation of the improved needle-catheter assembly as a whole. Also, on the top surface 34 are a plurality of contoured projections 32, intended for use as finger grips. In contrast, the sidewall surfaces 36 and the bottom surface 38 typically are smooth in order to accommodate the axial sliding movements of the needle housing unit 50 as described hereinafter.

In addition, as shown by FIGS. 5A and 5B, disposed on the top surface 34 of the needle-safety container 30 is a sized solid tab member 40, which appears in substantially rectangular form in this embodiment. The sized tab member 40, however, is not located directly at the forward end 31. Instead, the solid tab member 40 lies at a prechosen aligned position which is adjacent to, but is axially removed a short distance from the forward end 31 itself.

FIGS. 5B and 5C respectively illustrate the discrete bottom surface 38 of the elongated needle-safety container 30. As seen therein, the bottom surface 38 is split and purposely cut to provide a series of pre-positioned, aligned and geometrically sized cutouts for interaction with the guide member 80 of the needle housing unit 50. These aligned cutouts include a pair of contoured slots 43a and 43b lying adjacent to the forward end 31; an intersecting narrow median groove 45 extending along the middle of the bottom surface 38 and ending as a pair of bisected solid arms 47; and a contiguous wide terminal end recess 49 which lies adjacent to, but is spatially removed from, the terminal (proximal) end 33.

This spatial arrangement and prechosen alignment positioning thus generates two different and distinguishable zoned divisions within the article, which together constitute the elongated needle-safety container 30 as a whole. These zoned divisions are: a discrete collar zone 42 comprising the frontal end 31, the adjacently disposed and positionally aligned solid tab member 40, and the contoured slots 43a and 43b disposed on the bottom surface of the article; and an axial length zone 44 which begins adjacent to the collar zone 42, includes the narrow median groove 45, the bisected solid arms 47 and the wide terminal recess 49, and continues rearward to the terminal (proximal) end 33.

It is expected and intended that the downwardly extended wafer-like guide member 80 (joined to both the flash chamber 70 and the extended body section 56) will be of sufficient girth and spatial orientation to fit into and pass through the perimeter edges and spaced gap of the contoured parallel slots 43a and 43b as well as lie within the gap volume of the intersecting narrow median groove 45 respectively; and be contained by the perimeter edges of these pre-positioned cutouts as the guide member 80 is moved either radially within the collar zone 42 as part of the needle restraining process or linearly within the axial length zone 44 as a consequence of sliding the needle housing unit 50 over the needle-safety container 30.

In contrast, the bisected solid arms 47 and the wider spaced gap of the terminal end recess 47 serve as the pre-positioned cutouts and securing-in-place structures for capturing and holding the guide member 80 at the terminal (proximal) end 33. Consequently, when the guide member 80 becomes axially moved into that part of the bottom surface 38 where the bisected solid arms 47 and the terminal end recess 49 lie, the guide member 80 will easily pass between the bisected solid arms 47 and become fixed (or secured-in-place) within the wide gap space of the terminal end recess 49.

### A SECOND PREFERRED EMBODIMENT OF THE NEEDLE SAFETY CONTAINER

A second preferred embodiment of the needle-safety container is illustrated by FIGS. 6A, 68 and 6C respectively. FIG. 6A presents this second embodiment in a frontal, perspective view; FIG. 6B shows the article in cross-sectional view; and FIG. 6C provides a bottom view of the structure. Clearly, FIGS. 6A-6C respectively are markedly similar and comparable to the first embodiment shown by FIGS. 5A-5C.

Accordingly, a unified, contiguous needle-safety container appears as a hollow, cylinder-shaped article having an internal lumen; and is intended to be used in a needle-catheter assembly when positioned axially along its length as shown by FIG. 6A. The contiguous needle-safety container 130 presents a top surface 134, two sidewall surfaces 136, and a bottom surface 138; as well as a forward (distal) end 131, an internal lumen 135, and a terminal (proximal) end 133.

The forward end 131 of the article shown in FIG. 6A is intended to be engaged with and is preferably joined to the tapered protective tip 60 of the needle-catheter assembly [see FIGS. 1 and 2]. Also, in this second embodiment, the frontal end 131 also optionally includes a trio of supporting ledges 141 disposed within the internal lumen 135, and optionally are expected to hold a medial disc (not shown) for support of the piercing needle over its linear length. Similarly, situated on the top surface 134 of the contiguous article are a plurality of contoured projections 132, intended for use as finger grips. Also as before, the sidewall surfaces 136 and the bottom surface 138 typically are smooth in order to accommodate the axial sliding movements of the mounted needle housing unit 50 as described herein.

However, as shown by FIGS. 6A and 6B, the spatial arrangement of and structural construction for this second preferred embodiment differs in that it employs two separate and distinct cylindrically-shaped segments which are contiguously aligned and joined together axially to form a unified, contiguous needle-safety container 130 as a whole. These separate segments and discrete component parts are: a rotable at will collar segment 142; and a stationary (non-rotable) linear segment 144.

The rotable at will collar segment 142 comprises the forward end and the adjacently positionally solid tab member 140 which is disposed on the exterior surface of the collar segment, but lies removed a prechosen (short) distance from the forward end 131. The collar segment 142 is rotable on-demand as an individual and distinct entity while remaining axially aligned and joined to the adjacent linear segment 144. Thus, the solid tab member 40 on the collar segment 142 can be radially rotated independently and at will either to the left side or the right side of the adjacently located linear segment 144 freely, repeatedly, and without rotational hindrance or difficulty of movement.

In contrast, the adjacently located linear segment 144 remains non-rotable, i.e. stationary, as the collar segment 142 is rotated; and the linear segment 144 remains in an aligned axial orientation at all times despite the occurrence of a rotation for the collar segment, with the resulting radial displacement of the tab member 140.

FIG. 6C shows the contiguous bottom surface 138 of the unified needle-safety container 130. As seen therein, the contiguous bottom surface 138 is formed by the juncture of the collar segment 142 and the linear segment 144 collectively; and the contiguous bottom surface 138 is purposely incised to provide a series of pre-positioned and geometrically sized cutouts which will interact with the guide member 80 of the needle housing unit 50. Lying within the collar segment 142 are a pair of contoured parallel slits 143a and 143b which are disposed adjacent to the forward end 131 of the contiguous shell. Similarly, lying within the bottom of the linear segment 144 are an intersecting narrow median groove 145, a pair of bisected solid arms 147, and the wider terminal end recess 149 which lies adjacent to, but removed from, the terminal end 133 of the contiguous needle-safety container 130.

Also, as stated previously herein, it is expected and intended that the downwardly extended guide member 80 (attached to the flash chamber 70 and the bottom of the extended body section 56) will be of sufficient size and spatial orientation to fit into the spatial volume of the contoured parallel slots 143a and 143b of the collar segment 142; as well as pass through and slide along the gap space of the intersecting narrow median groove 145 of the linear segment 144. The guide member 80 will be held by and contained between these perimeter edges as the guide member is moved either radially within the collar segment 142 as part of the needle restraining process, or is moved axially within the linear segment 144 as a consequence of sliding the needle housing unit 50 over the contiguous needle-safety container 130.

Also as before, the distance between the perimeter edges of the wider spaced gap in the terminal end recess 149 is greater than the girth/thickness of the guide member 80 itself. Consequently, when the guide member is axially moved into that part of the contiguous bottom surface 138 where the bisected solid arms 147 lie, the guide member 80 will easily pass through the bisected arms and then become secured-in-place when resting partially within the wider gap space of the terminal end recess 149.

### B. The Needle Housing Unit

The modified needle housing unit provides a preferred, but optional, component part of the needle restraining structure and mechanism constituting the present invention, which can be used with both the first and the second preferred embodiments of the needle-safety container described previously herein.

A preferred embodiment of the modified needle housing unit is illustrated by FIGS. 7A-7D respectively. Accordingly, FIG. 7A shows a perspective frontal view of this article; FIG. 7B illustrates an overhead view of the unit; FIG. 7C presents a side view of the component; and FIG. 7D reveals a bottom view of the needle housing unit.

As shown by FIG. 7 as a whole, a modified needle housing unit 50 is a manufactured casing adapted for mounting upon and axial movement at will over the rotable needle-safety container illustrated previously herein by FIGS. 5 and 6 respectively. The needle housing unit 50 is a casing which has predetermined exterior and interior dimensions; has a prechosen geometric configuration which is preferably U-shaped and open at its top; and provides an internal spatial volume sufficient to house and encompass particular features and specific components.

As illustrated by FIGS. 7A-7D, the needle housing unit 50 is configured as a semi-tubular or U-shaped article and is completely open to the ambient air environment along its top. The needle housing unit 50 comprises two different structural elements: a spool section 52 and an extended body section 56. Each section and component part is described in detail hereinafter.

The Spool Section

The spool section 52 is a preferred, but optional, part of the needle retaining and locking mechanism; and is preferably structured as comprising a semi-circular flanged rib 53 and an adjacently disposed tab-engaging segment (or roll) 54 having at least one, and preferably two, sized notches (or slits) 55. Typically, in the preferred embodiment, the twice-notched tab-engaging segment 54 is rounded in shape and has a reduced girth/diameter with respect to the girth/diameter of the semi-circular flanged rib 53. Also, the sized notches (or slits) 55 existing within the rounded segment (or roll) 54 are dimensioned to provide a fixed gap volume and configuration sufficient to accommodate and engage on-demand the thickness and solid substance of the rotable tab member disposed on the exterior surface of the needle-safety container.

In addition, for needle retaining structure and locking mechanism purposes, the spool section 52 is purposely dimensioned and configured to provide a snug fit overlay around the collar zone (or its counterpart, the collar segment) at the forward end of the needle-safety container—such that the solid tab member then becomes spatially aligned with the rounded tab-engaging segment 54 and the sized notches 55 and has an unimpeded capability for on-demand radial rotation and at-will movement into friction engagement with either of the sized notches 55 in the spool section 52 of the needle housing unit 50. The radial orientation and spatial alignment requirements for the spool section 52 as a whole with respect to the forward end and the tab member of the needle-safety container are therefore important attributes and features in the preferred embodiment of the needle housing unit 50.

Accordingly, by this structural interplay and alignment of component parts, when the solid tab member is purposely radially rotated to either side (left or right), the tab member will become engaged, retained and locked in position within the gap volume of a notch 55 in the spool section 52 of the needle housing unit 50. Conversely, if and when the notch-engaged tab member is radially rotated in the opposite direction, the tab member will become disengaged and be released from the gap volume of notch 55 in the spool section 52 of the needle housing unit 50; and concomitantly will become free and rotable at will to regain its former top-oriented position.

The Extended Body Section

The semi-tubular configuration of the extended body section 56 of the needle housing unit 50 is conventionally known; and serves as a centrally located needle support, needle retractor, and means for securing-in-place the full length of the piercing needle as it recedes from the cannula lumen into the interior volume of the elongated needle-safety container. For ease in achieving this purpose, attached to opposing sides of the extended body section 56 are a pair of molded side panels 58a and 58b which provide easy-grip surfaces for holding and sliding the entire needle housing unit 50 over the axial length of the needle-safety chamber in either axial direction at will. Also positioned and contained entirely within the interior spatial volume of the needle housing unit 50 are a discrete flash chamber 70, a slidable guide member 80, and a needle hub 90. Each of these internally contained elements serves a different purpose.

### The Flash Chamber

The flash chamber 70 comprises a closed capsule 72 which has an internal air volume zone 74 at its front and is sealed via a microporous plug 76 at its rear. The primary function of the flash chamber 70 is to hold, support and encapsulate the proximal end of the piercing needle 14. A secondary function is to absorb such fluid (blood) as flows through the piercing needle during use.

Note therefore that, as shown by FIGS. 2 and 7B-7D respectively, the piercing needle 14 extends from within the lumen of the hollow cannula 10; passes through the catheter hub 20 and the needle guard 60; and is attached to the flash chamber 70 such that the proximal end of the piecing needle 14 terminates and lies entirely within the enclosed spatial volume 74 of the flash chamber 70.

### The Guide Member

The guide member 80 is also a conventionally known element. The primary purpose and function of the movable guide member 80 is two-fold: First, the flash chamber 70 is joined directly to the guide member 80; and these components always move together in unison and in tandem. Typically, the top of the guide member 80 is adjacently positioned, disposed upon, and joined to the bottom of the flash chamber 70; while the bottom of the guide member 80 is typically joined to an interior surface of the extended body section 56 of the needle housing unit 50. In this manner, both the flash chamber 70 and the guide member 80 remain in a fixed and constant position within the interior spatial arrangement of the needle housing unit 50, even though the needle housing unit itself is mobile with respect to the needle-safety container.

Second, the guide member 80 serves conventionally as an alignment aid which is used as the capture means and securing-in-place fixture for the piercing needle 14 when it is retracted and drawn rearward into the interior volume of the elongated needle-safety container. Thus, the lower portion of the guide member 80 passes through and moves axially along and within the gap space of the pre-positioned narrow median groove existing in the bottom surface of the elongated needle-safety container; can pass through the bisected solid arms; and will be ultimately become captured and secured-in-place by the bisected arms when lying partially within the wider terminal end recess of the needle-safety container.

To achieve these purposes, the guide member 80 has three distinct structural aspects, which are best illustrated by FIG. 7C. These are: a thin solid support and alignment base 82; a configured aperture, seen as a rectangular-shaped opening in FIG. 7C; and a narrow solid post 86. The support and alignment base 82 primarily acts as the substantive juncture and physical connection between the flash chamber 70 and the bottom of the extended body section 56; it provides the aligned guidance function. The configured aperture 84 and the solid post 86 serve in concert as the tangible entity and structural means by which the needle housing unit 50 as a whole is captured and secured-in-place via the bisected solid arms and the terminal end recess in the bottom surface of the needle-safety container shell described previously herein.

Accordingly, when the piercing needle 14 is placed in a retained and locked position on-demand by rotating the solid tab member and engaging a sized notch 55 in the spool section 52, the guide member 80 as a whole will also become radially rotated and move into the open space provided by one of the contoured parallel slots in the bottom of the needle-safety container. Subsequently, when the tab member is rotably disengaged from the sized notch 55 of the spool section 52—the guide member 80 also becomes concomitantly released from the contour slot of the needle safety container and moves back into the medial axis of the apparatus. Then, when the user then begins to retract the piercing needle rearward, the guide member 80, via a sliding movement, moves rearward within the intersecting narrow median groove in the bottom surface of the needle-safety container; and ultimately, becomes secure-in-place by passing through the solid bisected arms and then being held within the wider gap space of the terminal end recess located at the terminal (proximal) end of the needle-safety container. The consequence of the guide member 80 being secured-in-place in this manner is that the piercing needle becomes concomitantly placed in a non-movable, fixed setting.

### The Needle Hub

The needle hub 90 is typically positioned at the front of the extended body section 56, immediately rearward and adjacent to the spool section 52. The needle hub 90 is preferably a circular solid disc having a sized aperture 92 at its center. The aperture 92 is sufficiently large in diameter to allow one end of the piercing needle 14 to pass there through for subsequent juncture with and to the flash chamber 70. After one end of the piercing needle 14 has been permanently joined (in any conventionally known manner) to the flash chamber 70, the circular needle hub 90 serves to support the medial length of the piercing needle 14 during each and throughout all of its intended functions and actions in the catheter assembly.

### III. Engaging and Disengaging the Needle Restraining and Locking Mechanism On-Demand

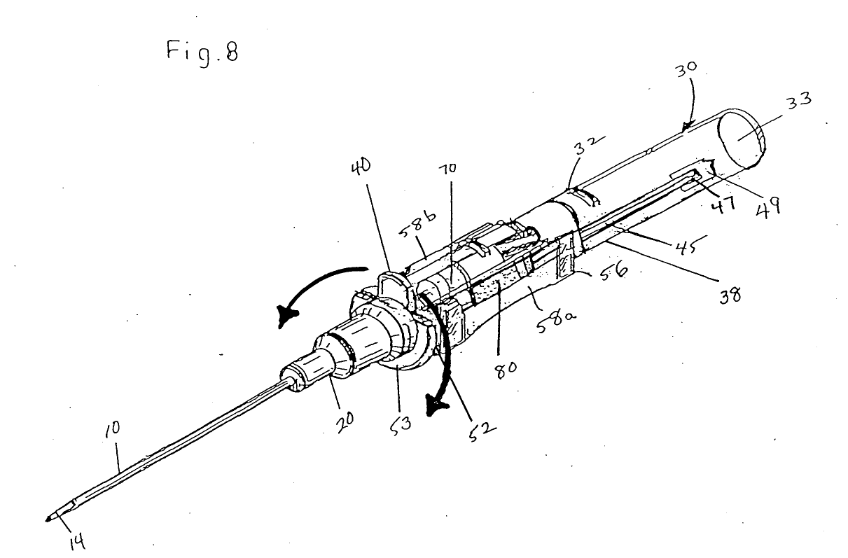

The means and manner of engaging and disengaging the needle restraining and locking mechanism comprising the present invention is illustrated by FIGS. 8-11 respectively. For clarity and ease of description, the needle-catheter assembly appearing in FIGS. 8-11 is shown as that first preferred embodiment previously described herein and illustrated by FIGS. 1-5 and 7 respectively.

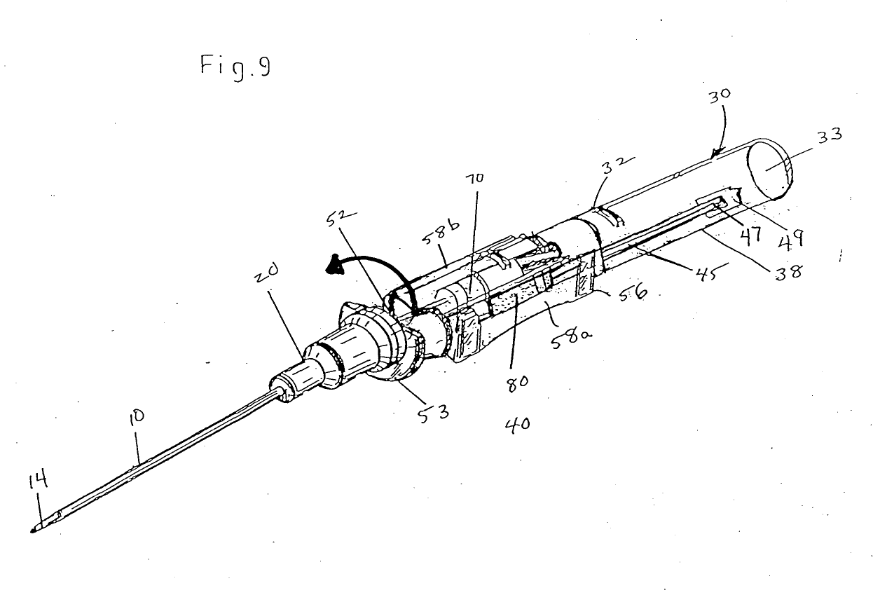

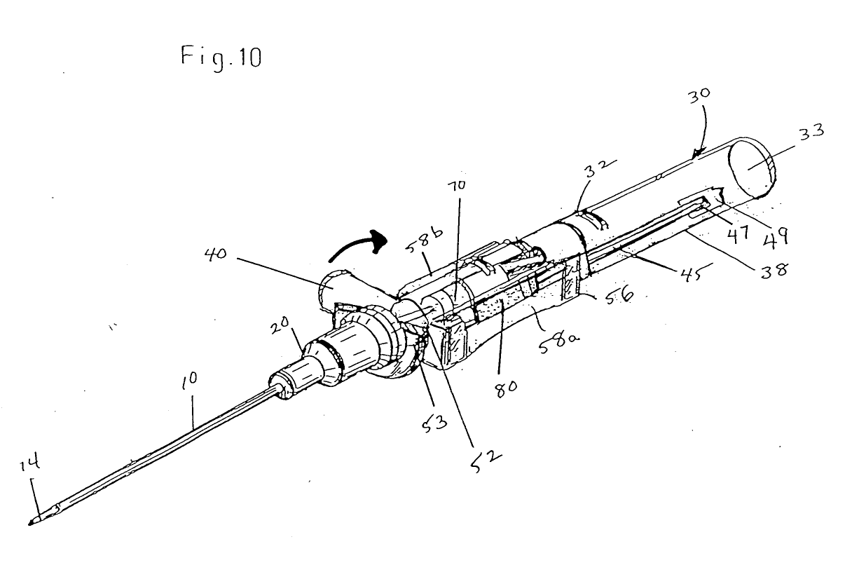

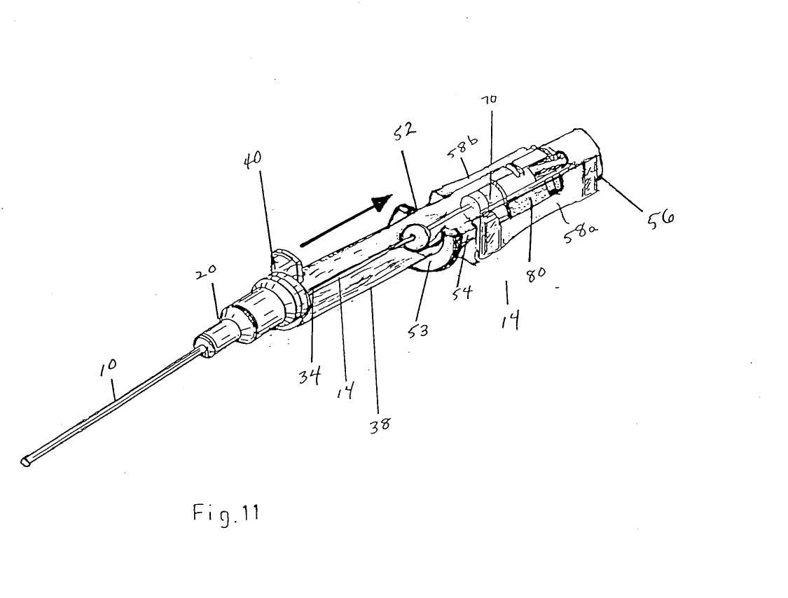

The needle-catheter assembly 2 is properly arranged and fitted together for immediate use as shown by FIG. 8. As seen therein, the solid tab member 40 of the elongated needle-safety container 30 is aligned with and lies adjacent to the spool section 52 of the needle housing unit 50, which is seen as mounted on and over the elongated needle-safety container 30. The semi-circular flanged rib 53 lies immediately forward of the tab member 40; and each of the sized notches 55 of the tab-engaging segment 54 are in radial alignment with and are situated immediately to the side and below the tab member 40 itself. Thus, whenever the need or desire of the user dictates, the tab member 40 can be radially manipulated and rotated at will either to the left or the right oriented direction (as shown by the arrows) for direct frictional engagement with the gap space of the sized notches 55 in the spool section 52 of the needle housing unit 50. Such manipulation and radial rotation on-demand results in the said tab member 40 being radially moved into direct contact with a notch 55; becoming engaged and retained by a notch 55; and being held and locked in position within the gap space of a notch 55 for any desired period of time. This result and outcome is shown by FIGS. 9 and 10 individually.

FIG. 9 illustrates a left-handed radial rotation of the tab member 40, which has then become engaged, retained and locked in position within the left-sided notch 55 in the spool section 52 of the mounted needle housing unit 50. The semi-circular flanged rib 53 aids in the restraint of the tab member 40; and also helps to prevent any accidental disengagement or inadvertent release of the engaged tab member 40 from the left-sided notch 55.

Similarly, FIG. 10 illustrates a right-handed radial rotation of the tab member 40, which has then become retained and locked in positioned within the right-sided notch 55 of the spool section 54. The semi-circular flanged rib 53 again aids in the restraint of the tab member 40 and also prevents any accidental disengagement or inadvertent release of the radially rotated tab member 40 from the right-sided notch 55.

Once the tab member 40 has been radially rotated, retained and locked into an engaged position within a notch 55 in the spool section 52 of the mounted needle housing unit 50 in the manner illustrated by either FIG. 9 or FIG. 10 respectively, the consequential and concomitant result of such tab member radial rotation and notch engagement manipulation is that the needle housing unit 50 in its entirety becomes immobilized. Such immobility, in that the whole of the needle housing unit 50 is completely restrained from any movement whatsoever—in turn, concomitantly causes and consequently insures that none of the individual components and features lying within the interior spatial volume of the extended body section 56 can either be moved axially or be altered in position with respect to all the other components of the needle-catheter assembly.

This intentionally generated state of immobility pertains in particular to the user's ability to move the piercing needle 14 (then lying within the lumen of the cannula 10) in either axial direction; and especially denies the user of any capability or power to retract (whether by accident or intention) the piercing needle 14 then lying within the cannula 10, presumably in the pierced skin of a patient. Thus, so long as the tab member 40 remains engaged, retained and locked within a notch 55 of the spool section 52, the piercing needle 14 then disposed co-axially within the cannula 10 cannot and will not become unintentionally moved or be inadvertently retracted. This is precisely what the user of this improved needle-catheter assembly will encounter—if and when the user fails to pierce and enter a blood vessel (vein) on his initial attempt to insert an catheter and then desires to withdraw the skin-embedded piercing needle from the patient in order to make a second (or multiple) further attempt at intravenous catheterization.

FIGS. 9 and 10 respectively therefore show the needle-catheter assembly in the needle restrained mode; and show the needle-catheter assembly as it should be employed when making any attempt at piercing a patient's vein for intravenous catheterization. Subsequently, if and when a chosen blood vessel in the patient is successfully pierced (no matter how many attempts are required before achieving actual success), then the retained and locked tab member can easily be disengaged and released at will by exerting a reverse direction radial rotation—as illustrated by the arrows appearing in FIGS. 9 and 10. This reverse radial rotation of the tab member will result in the earlier orientation shown in FIG. 8; and allow a free movement, a repositioning, and a full retraction of the piercing needle from within the cannula rearward into the interior spatial volume of the elongated needle-safety container, whenever the user chooses to slide the mounted needle housing unit 50 axially rearward.

The voluntary decision of the user and the resulting consequence of choosing to slide the needle housing unit 50 axially rearward is illustrated by FIG. 11. As shown therein, the needle housing unit 50 has been moved/slided axially rearward over the elongated length of the needle-safety container 30 to the maximum extent possible; and is seen as having reached the terminal (proximal) end 33 of the shell container. In this location, the piercing needle 14 has been fully withdrawn from the cannula lumen and now lies completely encompassed within the interior volume of the shell constituting the needle-safety container 30; and all the elements lying within the needle housing unit 50 (particularly the end of the piercing needle joined to the flash chamber) have become secured-in-place at this specific location as a consequence of the guide member 80 having entered and been secured within the wide gap space of the terminal end recess in the bottom surface of the needle-safety container. Accordingly, FIG. 11 shows the ultimate final arrangement and secured-in-place positioning for the catheter assembly after a successful intravenous catheterization of a blood vessel in a patient has been achieved.

The present invention is not to be restricted in scope nor limited in form except by the claims appended hereto.

What I claim is:

1. An on-demand needle retaining and locking mechanism for use in an intravenous needle-catheter assembly, said mechanism comprising:
a rotable on-demand needle-safety container comprised of
(i) an elongated shell having at least one discrete wall and being of predetermined dimensions and configuration,
(ii) an open end in said shell adapted for passage there through of a piercing needle,
(iii) an internal spatial volume within said shell sufficient for containing and securing the entirety of a piercing needle,
(iv) a sized tab member disposed on an exterior surface of said shell at a prechosen aligned position adjacent to, but axially removed from, said open end, and
(vi) a plurality of pre-positioned radial and axial cutouts in said wall of said shell wherein at least one of said cutouts is radially positioned and aligned with said tab member; and
a needle housing unit adapted for mounting upon and axial movement at will over said rotable needle-safety container, said needle housing unit being comprised of
(a) a casing of predetermined dimensions, configuration, and overall spatial volume,
(b) a flash chamber for holding one end of a piercing needle, and
(c) a guide member sized for aligned radial movement at will into and out said radially positioned cutout and for aligned axial movement through said axial cutout in said wall of said needle-safety container.

2. An on-demand needle retaining and locking mechanism for use in an intravenous needle-catheter assembly, said mechanism comprising:
a needle-safety container comprised of
a linear shell
(i) having at least one discrete wall and being of predetermined dimensions and configuration,
(ii) having an open end in said shell adapted for passage there through of a piercing needle, and
(iii) having an internal spatial volume sufficient for containing and securing a piercing needle,
(iv) at least one pre-positioned axial cutout in said wall of said linear shell,
a hollow collar contiguously aligned with and rotably attached to an open end of said linear shell, said rotable collar
(1) having at least one wall and being of predetermined dimensions and configuration,
(2) having two open ends adapted for passage there through of a piercing needle,
(3) a solid tab member which is disposed on an exterior surface of said wall, and
(4) at least one pre-positioned radial cutout in said wall which is radially positioned and aligned with said solid tab member; and
a needle housing unit adapted for mounting upon and axial movement at will over said needle-safety container and said rotable collar, said needle housing unit being comprised of
(a) a casing of predetermined dimensions, configuration, and overall spatial volume,
(b) a flash chamber for holding one end of a piercing needle, and
(c) a guide member for aligned radial movement at will into and out of said radially positioned and aligned cutout of said rotable collar and for axial movement at will through said axial cutout of said linear shell of said needle-safety container.

3. The needle-catheter assembly as recited by claim 1 or 2 wherein said radially positioned and aligned cutout comprises at least one contoured slot.

4. The needle-catheter assembly as recited by claim 1 or 2 wherein said pre-positioned cutouts include at least one axial groove which is linearly aligned for passage of said guide member.

5. The needle-catheter assembly as recited by claim 1 or 2 wherein said needle housing further comprises
a configured spool section comprising a tab-engagement segment and at least one sized notch for on-demand engagement with said tab member of said needle-safety container, said spool portion being alignable at will with said tab member and being able to engage, retain, and disengage said tab member of said needle-safety container on-demand; and
an extended body section

6. The needle-catheter assembly as recited by claim 5 wherein said configured spool section includes a pair of sized notches.

7. In a needle-catheter assembly including a hollow cannula, a piercing needle disposed co-axially within the cannula, an adjacently positioned needle-safety container, and a needle housing moveably mounted on said needle-safety container, the improvement of an on-demand needle retaining and locking mechanism comprising:
a rotable on-demand, elongated needle-safety container including
(i) an elongated shell having at least one discrete wall and being of predetermined dimensions and configuration,
(ii) an open end in said shell adapted for passage there through of a piercing needle,
(iii) an internal spatial volume within said shell sufficient for containing and securing the entirety of a piercing needle, and
(iv) a solid tab member disposed on an exterior surface of said shell at a prechosen aligned position adjacent to, but axially removed from, said open end, and
(v) a plurality of pre-positioned cutouts in said wall of said shell wherein at least one of said cutouts is radially positioned and aligned with said tab member and at least another of said cutouts is axially positioned; and
a needle housing unit adapted for mounting upon and axial movement at will over said rotable needle-safety container, said needle housing unit including
(a) a casing of predetermined dimensions, configuration, and overall spatial volume,
(b) a configured spool section comprising a flanged rib and a tab-engagement segment, and at least one notch for on-demand engagement with said tab member of said needle-safety container, said spool portion being alignable at will with and being able to engage, retain and disengage said tab member of said rotable needle-safety container on-demand,
(c) an extended body section, and
(d) a flash chamber for holding one end of a piercing needle, and
(e) a guide member for aligned radial and axial movement at will through said pre-positioned cutouts in said wall of said needle-safety container.

8. In a needle-catheter assembly including a hollow cannula, a piercing needle disposed co-axially within the cannula, an adjacently positioned needle-safety container, and a needle housing moveably mounted on said needle-safety container, the improvement of an on-demand needle retaining and locking mechanism comprising:
a needle-safety container including
a linear shell
(i) having at least one discrete wall and being of predetermined dimensions and configuration,
(ii) having an open end in said shell adapted for passage there through of a piercing needle, and
(iii) having an internal spatial volume sufficient for containing and securing a piercing needle,
(iv) at least one pre-positioned axial cutout in said wall of said linear shell,
a hollow collar contiguously aligned with and rotably attached to an open end of said linear shell, said rotable collar including
(1) at least one wall and being of predetermined dimensions and configuration,
(2) two open ends adapted for passage there through of a piercing needle, and
(3) a least one radially positioned and aligned cutout in said wall, and
(5) a solid tab member disposed on an exterior surface of said wall of said rotable collar at a position adjacent to, but spatially removed from, an end of said rotable collar, said position being radially aligned with said cutout in said wall; and
a needle housing unit adapted for mounting upon and axial movement at will over said needle-safety container and said rotable collar, said needle housing unit including
(a) a casing of predetermined dimensions, configuration, and overall spatial volume,
(b) a configured spool section comprising a tab-engagement segment, and at least one sized notch for on-demand engagement with said tab member of said rotable collar, said spool section being alignable at will with and being able to engage, retain and disengage said tab member of said rotable collar on-demand,
(c) an extended body section, and
(d) a flash chamber for holding one end of a piercing needle, and
(e) a guide member for aligned radial and axial movement at will through said pre-positioned cutouts of said rotable collar and of said linear shell of said needle-safety container.
